(19) United States
(12) Patent Application Publication
(10) Pub. No.: US 2024/0010992 A1
(43) Pub. Date: Jan. 11, 2024

(54) COMPOSITIONS AND METHODS TO IMPROVE IPSC-EC FUNCTION

(71) Applicant: Northwestem University, Evanston, IL (US)

(72) Inventors: Katherine E. Hekman, Atlanta, GA (US); Jason A. Wertheim, Tucson, AZ (US); Congcong He, Chicago, IL (US)

(21) Appl. No.: 18/350,161

(22) Filed: Jul. 11, 2023

Related U.S. Application Data
(60) Provisional application No. 63/388,129, filed on Jul. 11, 2022.
(60) Provisional application No. 63/401,014, filed on Aug. 25, 2022.

(51) Int. Cl. C12N 5/074; A61K 35/545; A61F 2/06; A61L 27/50

(52) U.S. Cl. CPC C12N 5/0696; A61K 35/545; A61F 2/06; A61L 27/507; C12N 2501/727

(57) ABSTRACT

Provided herein are compositions and methods to promote longevity of induced pluripotent stem cell (iPSC)-derived endothelial cells (ECs) via autophagy. In particular, provided herein are AMP-activated protein kinase (AMPK) activators, such as ginsenosides (e.g., Rg2), and use thereof to increase longevity of iPSC-ECs by stimulating mTOR-independent ULK1-mediated autophagy.

### CROSS-REFERENCE TO RELATED APPLICATIONS

This application claims the benefit of U.S. Provisional Patent Application No. 63/388,129, filed on Jul. 11, 2022, and U.S. Provisional Patent Application No. 63/401,014, filed on Aug. 25, 2022, both of which are incorporated by reference herein.

### STATEMENT REGARDING FEDERAL FUNDING

This invention was made with Government support under HL137292, DK113168, DK113170, and DK123447 awarded by the National Institutes of Health. The government has certain rights in the invention.

### FIELD

Provided herein are compositions and methods to promote longevity of induced pluripotent stem cell (iPSC)-derived endothelial cells (ECs) via autophagy. In particular, provided herein are AMP-activated protein kinase (AMPK) activators, such as ginsenosides (e.g., Rg2), and use thereof to increase longevity of iPSC-ECs by stimulating mTOR-independent ULK1-mediated autophagy.

### BACKGROUND

Derivation of endothelial cells and vascular tissue from induced pluripotent stem cells (iPSCs) represents a powerful tool for the study, diagnosis, and treatment of peripheral arterial disease. At present this tool remains limited by the loss of mature cellular phenotypes and degenerative changes within the cellular progeny derived from iPSCs, ultimately culminating in senescence [1, 2]. Modulation of this senescence may lead to improved functionality and a more robust and durable phenotype of the resulting terminally differentiated progeny.

Endothelial cells differentiated from human iPSCs and from human embryonic stem cells via embryoid bodies typically do not retain mature endothelial cell surface markers for more than 2 weeks [3]. More recently, generating iPSC-ECs as 3-dimensional organoids appears to improve the longevity of the resulting cells, however the applicability of this method to complex tissue and organ engineering remains to be demonstrated [4]. Multiple stimulators of autophagy activity, such as lentiviral-mediated overexpression of the longevity gene sirtuinl (SIRT1) as well as nutrient starvation, each attenuate premature senescence in iPSC-derived endothelial cells (iPSC-ECs) in conventional approaches to EC differentiation [5, 6]. However, the precise mechanisms by which these interventions attenuate premature senescence are poorly understood.

### SUMMARY

Provided herein are compositions and methods to promote longevity of induced pluripotent stem cell (iPSC)-derived endothelial cells (ECs) via autophagy. In particular, provided herein are AMP-activated protein kinase (AMPK) activators, such as ginsenosides (e.g., Rg2), and use thereof to increase longevity of iPSC-ECs by stimulating mTOR-independent ULK1-mediated autophagy.

In some embodiments, provided herein are methods of increasing the longevity of a cell population comprising stimulating mTOR-independent ULK1-mediated autophagy in the cells. In some embodiments, the cell population comprises cells derived from induced pluripotent stem cells (iPSCs). In some embodiments, the cell population comprises induced pluripotent stem cell-derived endothelial cells (iPSC-ECs). In some embodiments, methods comprise administering an agent to the cell population to stimulate mTOR independent ULK1-mediated autophagy. In some embodiments, the agent is a small molecule. In some embodiments, the cell population is cultured in the presence of the agent. In some embodiments, the agent is an adenosine monophosphate kinase (AMPK) activator. In some embodiments, the AMPK activator is an indirect activator. In some embodiments, the indirect activator is Rg2. In some embodiments, the AMPK activator is a direct activator. In some embodiments, the agent is an activator of unc-51-like autophagy activating kinase 1 (ULK1).

In some embodiments, provided herein are cell populations comprising cells that have been cultured in the presence of an agent capable of increasing the longevity of a cell population comprising stimulating mTOR-independent ULK1-mediated autophagy. In some embodiments, the agent is an adenosine monophosphate kinase (AMPK) activator. In some embodiments, the agent is an activator of unc-51-like autophagy activating kinase 1 (ULK1). In some embodiments, the cells are iPSC-derived cells. In some embodiments, the cells are iPSC-ECs.

In some embodiments, provided herein are medical devices comprising cells treated with an agent capable of increasing the longevity of a cell population comprising stimulating mTOR-independent ULK1-mediated autophagy. In some embodiments, the device is a vascular graft.

In some embodiments, provided herein are bioengineered organ comprising cells treated with an agent capable of increasing the longevity of a cell population comprising stimulating mTOR-independent ULK1-mediated autophagy.

In some embodiments, provided herein are methods of treating a condition in a subject comprising administering cells treated with an agent capable of increasing the longevity of a cell population comprising stimulating mTOR-independent ULK1-mediated autophagy. In some embodiments, the condition is ischemic tissue and iPSC-ECs are administered to promote angiogenesis.

In some embodiments, provided herein are compositions comprising (a) cell growth media components and (b) and agent capable of stimulating mTOR-independent ULK1-mediated autophagy in the cells. In some embodiments, the agent is an adenosine monophosphate kinase (AMPK) activator. In some embodiments, the agent is an activator of unc-51-like autophagy activating kinase 1 (ULK1).

### BRIEF DESCRIPTION OF THE DRAWINGS

FIG. 1A-B. Loss of CD144 and CD31 expression over serial passages correlated with a loss of cellular proliferation. (A) Immunofluorescence confirmed the loss of CD144 and CD31, as well typical morphology, over serial passages of purified iPSC-ECs. (B) iPSC-ECs demonstrated a decrease in the percent of cells proliferating by EdU labeling over serial passages in culture.

FIG. 2A-D. Autophagy plays a critical role and recycles mitochondria during the course of differentiation of Y6 cells. (A) In directed differentiation of Y6, total LC3-II decreased from Day 2 to 3 and increased from Day 4 to 5. (B) TEM images of Y6 during differentiation demonstrated a significant increase in number of autolysosomes from day 2 to day 3 (p<0.05), and illustrated the evolving mitochondrial morphology, from immature on Day 1 to complex and prototypical on Day 6. (C) TEM showed autolysosomes recycling mitochondria starting on day 3 of differentiation. (D) Representative image of a mature mitochondria on day 6 of differentiation.

FIG. 3A-E. Mitochondria accumulate over serial passages in ACS1028-ECs without a compensatory increase in recycling. (A) Top panel: MitoTracker staining of ACS1028 cells on each day of differentiation to ECs demonstrated that mitochondrial mass and morphology change over the course of differentiation. Middle panel: MitoTracker staining of each passage of ACS1028-ECs showed mitochondrial accumulation in later passages. Bottom panel:

Quantification of average MitoTracker signal per cell by fluorescence microscopy revealed a significant and greater than 20-fold increase in mitochondria in iPSC-ECs (p<0.05). (B) LC3-II expression remained stable throughout serial passages of Y6-ECs. (C-D) Representative TEM images of Y6-ECs from each passage showing a stable number of autolysosomes over serial passages. (E) Quantification of autolysosomes containing mitochondrial structures demonstrated a decrease in mitophagy in Y6-ECs compared to during differentiation (p<0.05).

FIG. 4A-C. Hypoxia reduced MitoTracker staining per ACS1028-EC cell at each passage but still resulted in loss of proliferation over serial passages. (A) Schematic diagram of hypoxia experiment. (B) 1.5% oxygen reduced MitoTracker red and green staining per ACS1028-EC cell compared to normoxic conditions at early passages, but cells ultimately accumulated mitochondria in later passages. (C) ACS1028-EC proliferation was improved under hypoxic conditions but still declined over serial passages.

FIG. 5A-C. Plant derivative Rg2 improved proliferation of ACS1028-ECs over serial passages. (A) EdU labeling of ACS1028-ECs treated with Rg2, rapamycin or DMSO as a vehicle control demonstrated improved proliferation with Rg2 treatment. (B) Quantification of EdU labeled cells demonstrated a statistically significant improvement in proliferation with Rg2 treatment compared to treatment with rapamycin, ML246, or mitophagy inducers Antimycin A or oligomycin. (C) The benefit to proliferation conferred by Rg2 was sustained through passage 3.

FIG. 6A-C. Ulkl kinase inhibitor SBI-0206965 blocked the survival benefit associated with increased LC3-II that was conferred by Rg2. (A) Manual counting of cells in light microscopy demonstrated a statistically significant reduction in cell survival in a dose-dependent manner when cells were co-treated with SBI-0206965 in the presence of Rg2. (B-C) Western blotting of ACS1028-EC lysate from serial passages treated with DMSO as a control, or with Rg2, or with Rg2 and various concentrations of SBI-0206965 demonstrated a significant increase in LC3-II expression with Rg2 treatment at passage 3 that is attenuated by co-treatment with SBI-0206965.

FIG. 7A-C. Induced pluripotent stem cells (iPSCs) successfully lost pluripotency markers and gained endothelial markers CD144 and CD31 during directed differentiation, but expression of these markers was not stable and disappeared after several passages in culture. (A) Flow cytometry demonstrated that pluripotency marker Nanog disappeared by Day 5 (left) and pluripotency marker Oct 4 began to decrease on Day 4 (right). Bar graph on the far right quantifies the progression of the acquisition of CD144 expression and loss of Nanog and Oct4 pluripotency markers during directed differentiation to endothelial cells. (B) Quantification of percent CD144 positive cells each day of differentiation by flow cytometry. (C) After purification of iPSC-derived endothelial cells (iPSC-ECs) on day 6 of differentiation, the iPSC-ECs began to lose CD144 expression over serial passages.

FIG. 8A-C. Autophagy recycles mitochondria during differentiation of ACS1028 to ECs. (A) In directed differentiation of ACS1028, total LC3-II decreased from Day 3 to 4 and increased from Day 5 to 6. (B) TEM images of ACS1028 during differentiation demonstrated a significant increase in number of autolysosomes from day 2 to day 3 (p<0.05). (C) TEM showed autolysosomes recycling mitochondria starting on day 3 of differentiation.

FIG. 9A-E. Mitochondria accumulate over serial passages in Y6-ECs without a compensatory increase in recycling. (A) Top panel: MitoTracker staining of Y6 cells on each day of differentiation to ECs demonstrated that mitochondrial mass and morphology change over the course of differentiation. Middle panel: MitoTracker staining of each passage of Y6-ECs showed mitochondrial accumulation in later passages. Bottom panel: Quantification of average MitoTracker signal per cell by fluorescence microscopy revealed a significant and greater than increase in mitochondria in Y6-ECs (p<0.05). (B) Purified ACS1028-derived ECs showed stable levels of LC3-II over serial passages. (C-E) TEM analysis of ACS1028-derived ECs demonstrated a decrease in mitophagy despite a comparable number of autolysosomes over serial passages compared to Day 6 of differentiation (p<0.05).

FIG. 10A-E. Hypoxia did not prevent loss of morphology and CD144/CD31 expression over serial passages. (A) Despite improvement in mitochondrial accumulation over serial passages, ACS1028-ECs still demonstrated a loss of typical morphology and expression of CD144 and CD31. (B) Y6-ECs also still demonstrated a loss of typical morphology and CD144/CD31 expression. (C) Hypoxia (1.5% oxygen) improved both total MitoTracker green and MitoTracker red signal per cell Y6-derived ECs, although mitochondria did continue to accumulate under hypoxic conditions. (D) Hypoxia improved the number of Y6-ECs detected per high power field in early passages. (E) Hypoxia also improved NO synthase activity per cell as detected by reduction of DAF-FM.

FIG. 11A-C. Mitophagy inducing drugs Antimycin A and oligomycin significantly decreased cell number as detected by total Hoechst signal by passage 1 at all tested concentrations. (A) Schematic diagram of mitophagy inducing drug experiment. (B-C) Mitophagy inducers at all concentrations decreased cell survival of ACS1028-derived ECs and Y6-derived ECs.

FIG. 12A-B. Mitophagy inducers Antimycin A and oligomycin accelerated the loss of CD144 and CD31 as well as typical morphology. Mitochondrial uncoupler FCCP also accelerated loss of mature endothelial cell phenotype, suggesting iPSC-ECs do not tolerate the loss of mitochondrial membrane potential. A) ACS1028. B) Y6.

FIG. 13A-C. Certain autophagy inducers as well as the ULK1 kinase inhibitor SBI-0206965 were not tolerated by purified ACS1028-ECs, and light microscopy demonstrated dose-dependent decreased survival of Rg2-treated iPSC-ECs with increasing doses of Ulkl kinase inhibitor. (A) Light microscopy demonstrating presence of cells under control and DMSO-vehicle control conditions, with no cells visible in the SBI-0206965, resveratrol, ML246, or spermidine conditions. (B) The conditions in (A) demonstrated no residual cells when incubated overnight with Trypan blue. (C) Light microscopy images demonstrated significantly fewer surviving cells per high power field with DMSO control versus with Rg2, and with cells co-treated with SBI-0206965 and Rg2.

FIG. 14. Passage 0 endothelial cells purified from ACS1028-ECs characterized by stitched CD144 labeled cells. Confocal microscopy demonstrating CD144 labeled endothelial cells forming confluent layers with clear boundaries indicative of endothelial junctions.

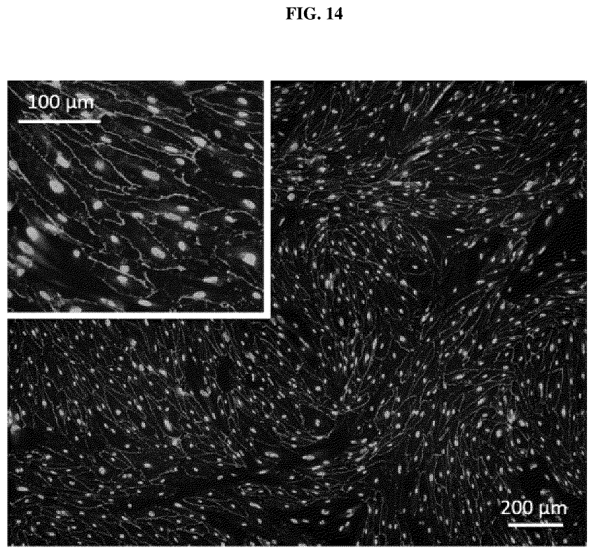

FIG. 15. Rg2 increases expression of longevity protein SIRT1 in a dose-dependent manner.

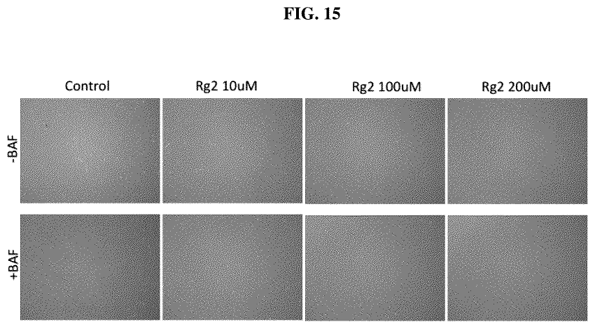

FIG. 16. Rg2 increases phosphorylation (activity) of endothelial nitric oxide synthase

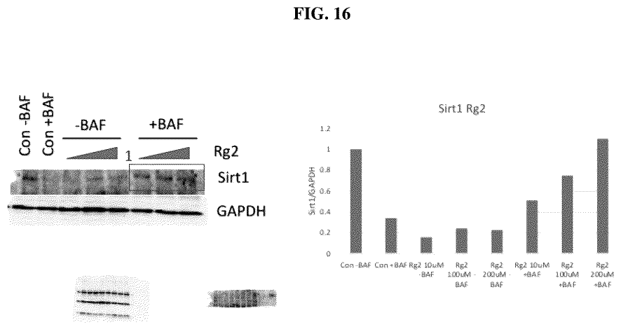

FIG. 17. Rg2 does not appear to induce apoptosis (no increase in cleaved caspase 3 at tested doses).

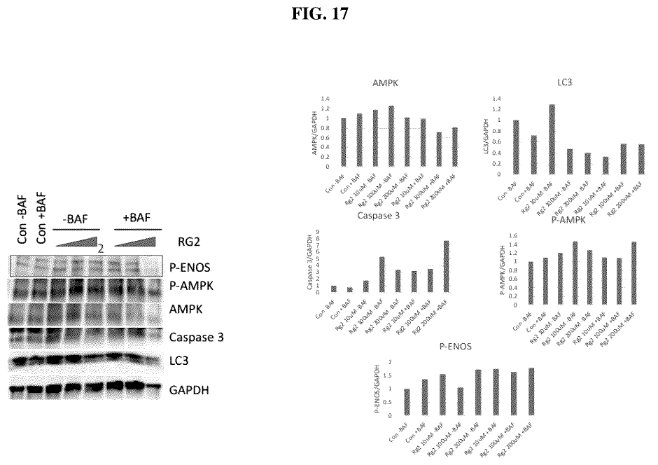

FIG. 18. Rg2 slightly increases Parkin (mitophagy marker) expression, under bafilomycin treated conditions to inhibit autophagosome-lysosome fusion.

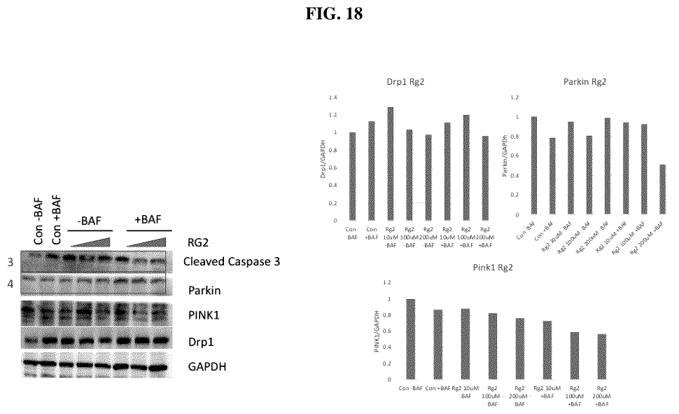

FIG. 19. Rg2 increase phosphorylation of Drp1 (mitochondrial fission) and expression of Opal (mitochondrial fusion).

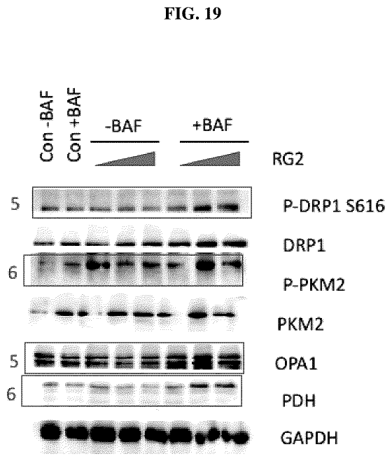

FIG. 20. Rg2 increases phosphorylation of PKM2 and expression of PDH, particularly under bafilomycin treated conditions to inhibit autophagosome-lysosome fusion.

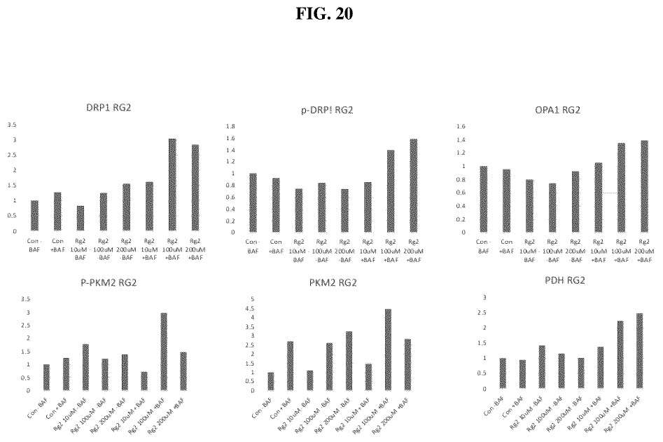

### DETAILED DESCRIPTION

Provided herein are compositions and methods to promote longevity of induced pluripotent stem cell (iPSC)-derived endothelial cells (ECs) via autophagy. In particular, provided herein are AMP-activated protein kinase (AMPK) activators, such as ginsenosides (e.g., Rg2), and use thereof to increase longevity of iPSC-ECs by stimulating mTOR-independent ULK1-mediated autophagy.

Experiments were conducted during development of embodiments herein to identify the impact of autophagy, that is, the process of cellular self-digestion that recycles intracellular components, during the differentiation and culture of iPSC-ECs. During the course of unraveling the role of autophagy in iPSC-ECs, experiments uncovered alternative targets for overcoming this premature proliferative senescence.

Stem cells are enabling an improved understanding of peripheral arterial disease, and stem cell-derived endothelial cells (ECs) present major advantages as a therapeutic modality. However, applications of induced pluripotent stem cell (iPSC)-derived ECs are limited by rapid loss of mature cellular function in culture. Experiments were conducted during development of embodiments herein to determine if changes in autophagy impact the phenotype and cellular proliferation of iPSC-ECs. Endothelial cells were differentiated from distinct induced pluripotent stem cell lines in 2D culture and purified for CD144 positive cells. Autophagy, mitochondrial morphology, and proliferation were characterized during differentiation and over serial passages in culture. Autophagy activity was stimulated during differentiation but stagnated in mature iPSC-ECs. Mitochondria remodeled through mitophagy during differentiation and demonstrated increasing membrane potential and mass through serial passages; however, these plateaued, coinciding with decreased proliferation. To evaluate for oxidative damage, iPSC-ECs were alternatively grown under hypoxic culture conditions; however, hypoxia only transiently improved proliferation. Stimulating mTOR-independent ULK1-mediated autophagy with a plant derivative AMP kinase activator Rg2 significantly improved proliferative capacity of iPSC-ECs over multiple passages. Autophagy played an active role in remodeling mitochondria during maturation from pluripotency to a terminally differentiated state. Autophagy failed to compensate for increasing mitochondrial mass over serial passages, which correlated with loss of proliferation in iPSC-ECs. Stimulating ULK1-kinase driven autophagy conferred improved proliferation and longevity over multiple passages in culture. In some embodiments, provided herein are compositions and methods of stimulating ULK1-kinase driven autophagy to overcome a major barrier limiting the use of iPSC-ECs for clinical and research applications.

The experiments conducted during development of embodiments herein highlight the importance of autophagy in the differentiation and phenotypic longevity of iPSC-ECs. Premature senescence is attenuated with treatment (e.g., via small molecule agents) to augment mTOR-independent ULK1-mediated autophagy, a method that, unlike viral transduction, is readily translated to clinical use.

In some embodiments, provided herein are methods of increasing the longevity of iPSC-ECs comprising stimulating mTOR-independent ULK1-mediated autophagy. In some embodiments, methods comprise administering an agent to the iPSC-ECs to stimulate mTOR independent ULK1-mediated autophagy. In some embodiments, the agent is an AMP kinase activator. In some embodiments, the agent is a peptide, antibody, antibody fragment, protein, nucleic acid, small molecule, etc. In some embodiments, the agent binds to an modifies (e.g., enhances, inhibits) the activity of a bioactive component of the cell (e.g., ULK1, AMPK, etc.). In some embodiments, the agent modifies (e.g., enhances, inhibits) expression of a bioactive component of the cell (e.g., ULK1, AMPK, etc.).

In some embodiments, the agent is a small molecule. In some embodiments, the agent is Rg2. Experiments conducted during development of embodiments herein demonstrate that addition of Rg2 to iPSC-ECs rescues their senescent phenotype and restores proliferation.

In some embodiments, methods are provided for increasing the longevity of a cell population by stimulating mTOR-independent ULK1-mediated autophagy. In some embodiments, the cells are iPSC-derived cells. In some embodiments, the cells are iPSC-endothelial cells or iPSC-smooth muscle cells. In particular embodiments, the cells are iPSC-derived endothelial cells. In some embodiments, the cells are iPSC-derived vascular endothelial cells. In other embodiments, the cells are iPSC-derived lymph endothelial cells.

In some embodiments, an agent is administered to a cell population (e.g., iPSC-derived cells, iPSC-ECs, etc.). In some embodiments, the agent is capable of stimulating mTOR-independent ULK1-mediated autophagy. In some embodiments, the agent is an activator of AMP-activated protein kinase (AMPK). In some embodiments, the agent does not inhibit the mammalian target of rapamycin (mTOR) signaling pathway.

In some embodiments, an AMPK activator is an indirect activator of AMPK. AMPK is activated by agents that cause AMP accumulation or calcium accumulation. Examples of suitable indirect activators of AMPK include biguanides (e.g., metformin), thiazolidinediones (e.g., rosiglitazone, pioglitazone, etc.), polyphenols (e.g., flavonoids, phenolic acid, polyphenolic amides, resveratrol, curcumin, lignans, etc.), ginsenosides (e.g., Rb1, Rb2, Rc, Re, Rg1, Rg2, Rg3, etc.), a-lipoic acid, a statin, hydrogen sulfide, etc.

In some embodiments, an AMPK activator is a direct activator of AMPK. Direct activators of AMPK bind directly to AMPK and cause AMPK activation without any significant change in cellular ATP, ADP or AMP levels. Instead, direct AMPK activators induce conformation changes in the AMPK complex, leading to activation, possibly through a direct interaction with a specific subunit of AMPK. Examples of suitable direct activators of AMPK include 5-aminoimidazole-4-carboxamide riboside, thienopyridone compounds (Cool et al. Cell Metab 2006; 3: 403-416.; incorporated by reference in its entirety), benzimidazole derivatives (Xiao et al. Nat Commun 2013; 4: 3017.; incorporated by reference in its entirety), salicylate, 5-(5-hydroxyl-isoxazol-3-yl)-furan-2-phosphonic acid and prodrugs thereof, PT-1 (Pang et al. J Biol Chem 2008; 283: 16051-16060.; incorporated by reference in its entirety), MT 63-78 (Debio0930) (Zadra et al. EMBO Mol Med 2014; 6: 519-538.: incorporated by reference in its entirety), etc.

In some embodiments, an AMPK activator is Rg2. In experiments conducted during development of embodiments herein, Rg2 demonstrated a potent effect on iPSC-EC proliferation in culture. The effect of Rg2 correlated with LC3-II levels and was mitigated by SBI-0206965, demonstrating that it is mediated through ULK1 kinase autophagy. However, this benefit was not reproduced with rapamycin treatment, indicating that Rg2 is conferring this benefit through mTOR-independent autophagy. Rg2 was previously demonstrated to act through AMP kinase activation [10]. Interruption of the ULK1-AMP kinase pathway has been demonstrated to disrupt mitophagy, [13]. Additionally, lentiviral mediated SIRT1 overexpression in iPSC-ECs enhanced longevity, as did nutrient starvation [5, 6]. The ULK1-AMP kinase pathway was also implicated in stabilization of SIRT1 by nitric oxide in HUVECs [24]. Experiments conducted during development of embodiments herein demonstrate a significant improvement in iPSC-EC proliferation and viability with ULK1-mediated autophagy when treated with Rg2.

In some embodiments, an agent is administered to a cell population is an ULK1 kinase activator. An example of a suitable ULK1 kinase activator is:

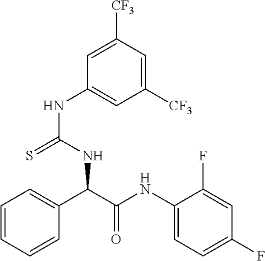 [SMILES: O=C(Nc1ccc(F)cc1F)[C@H](NC(=S)Nc1cc(C(F)(F)F)cc(C(F)(F)F)c1)c1ccccc1]

(Ouyang et al. J. Med. Chem. 2018, 61, 7, 2776-2792; incorporated by reference in its entirety),

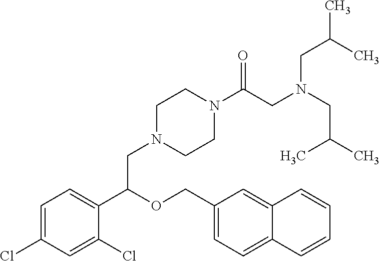 [SMILES: CC(C)CN(CC(=O)N1CCN(CC(OCc2ccc3ccccc3c2)c2ccc(Cl)cc2Cl)CC1)CC(C)C]

(Ouyang L, et al. J Med Chem. 2018 Apr. 12; 61(7):2776-2792.; incorporated by reference in its entirety); although other ULK1 kinase activators are within the scope herein.

In some embodiments, cells are cultured in/on media containing an agent described herein for enhancing cell longevity, preventing premature senescence, and/or improving cellular function. The cells may be of any suitable cell type (e.g., iPSC-derived cells (e.g., iPSC-ECs, etc.), etc.) and may be cultured in a cell-type appropriate media, as would be understood in the field. In some embodiments, cells are cultured in 2D or 3D culture, with solid or liquid media. In some embodiments, the agent described herein for enhancing cell longevity, preventing premature senescence, and/or improving cellular function is added to the media, is a component of the media, or is added directly to the cells.

In some embodiments, a composition comprising cell media components and an agent described herein is provided. Suitable cell media components may include, but are not limited to amino acids, vitamins, inorganic salts, glucose, serum, buffers, proteins (albumin, globulins, IgG, etc.).

Embodiments herein find use in, for example, engineering cellularized grafts (e.g., to treat peripheral artery disease), vascularizing bioengineered organs for transplantation, injecting to stimulate local angiogenesis in ischemic tissue, etc. In some embodiments, provided herein are vascular grafts containing or comprising cells that have been treated according to the embodiments described herein to enhance cell longevity, prevent premature senescence, and/or improve cellular function. In some embodiments, provided herein are bioengineered organs comprising the cells treated according to embodiments herein. In some embodiments, provided herein are methods of treating a condition in a subject comprising injecting or transplanting cells treated according to embodiments herein into the subject. In some embodiments, the type of cells administered is dependent on the condition to be treated (e.g., iPSC-ECs for stimulation of angiogenesis in ischemic tissue).

### EXPERIMENTAL

### Example 1

### Materials and Methods

### Cell Lines and Stem Cell Culture

The iPSC line ACS1028 was obtained from ATCC (ATCC-BXS0114) and were cultured on vitronectin coated plates (Thermo Fisher A31804) with E8 flex media (Gibco A2858501). The iPSC line Y6 was provided by the Yale University Stem Cell Center, and the cells were cultured on Matrigel coated plates (Fisher Scientific 08-774-552) with MtESR1 media (Stem Cell Technologies 85850). Passages 10-25 were used in all experiments. Cells were passaged with ReLESR (Stem Cell Technologies 05872) and plated with 10 μM rock inhibitor (R&D 1254) for 24 hours. Both lines were sent to IDEXX for STR validation, but neither existed in the repository. Routine cytogenetics and mycoplasma testing (Lonza LT07-318) were performed.

### Endothelial Cell Differentiation and Maintenance

Directed differentiation of pluripotent stem cells to endothelial cells was performed according to the published protocol, using CHIR09921 (R&D 4423), BMP4 (Peprotech 120-05), forskolin (Abcam ab120058), and VEGF165 (Peprotech 100-20) [7]. Endothelial cells were maintained in Vasculife VEGF media (Lifeline Cell Technology LL-0003). CD144 positive cells were separated by magnetic associated cell sorting (MACS) using CD144-conjugated magnetic beads (Miltenyi 130-097-857) in LS columns (Miltenyi 130-042-401). Purified iPSC-ECs were plated on fibronectin (Fisher Scientific CB-40008A) coated plates. iPSC-ECs were passaged with 0.025% trypsin and trypsin neutralizing solution (Lonza CC-5012 and CC-5002, respectively).

### Immunostaining

Immunostaining was performed by fixing cells in 4% paraformaldehyde for 15 minutes at room temperature and then washing with PBS. Cells were then permeabilized with 70% ice cold ethanol on ice and blocked with 0.5% BSA in PBS for 15 minutes at room temperature. Cells were then incubated overnight at 4° C. with the primary antibody in antibody dilution buffer (0.05% BSA in PBS) at the following concentrations: Nanog (Life Technologies PA1-097, 1:100), SSEA4 (Life Technologies 414000, 1:400), Sox2 (Life Technologies PA1-16968, 1:100), VE-cadherin/CD144 (Sigma V1514, 1:200), PECAM1/CD31 (Abcam ab187377, 1:100). The isotype control antibodies used in this study were mouse IgG1 (Abcam ab172730) and rabbit serum. Cells were washed with PBS and incubated with conjugated secondary antibodies at room temperature in the dark (Life Technologies, 1:300).

### Flow Cytometry

Cells were lifted with Accutase (Stem Cell Technologies 07922) and stained with blue fixable live/dead stain (Invitrogen L34961 or L23105). Cells were then fixed with 4% PFA for minutes at room temperature. For Nanog (BD Biosciences 560483) and Oct4 (Novus Biologicals NB100-2379) staining, cells were permeabilized with 0.1% Triton X-100 for 15 minutes on ice and blocked with 1% BSA/PBS on ice for 30 minutes. Cells were then incubated with the conjugated primary antibodies, 20 μL of antibody for 1×10⁶ cells in 100 μL of 1% BSA/PBS. For CD144 and CD31 (BD Biosciences 560410 and 560984, respectively), fixed cells were incubated with the conjugated primary antibody, 20 μL of antibody for 1×10⁶ cells in 100 μL of 1% BSA/PBS. Cells were then washed 3 times with 1% BSA/PBS. Flow cytometry was performed on the BD LSR Fortessa and analyzed in FloJo v10.

### Cell Proliferation Assay

Proliferating cells were labeled with EdU using the Invitrogen Click-iT microplate assay kit (Invitrogen C10214), without the use of the Amplex Ultra red step. Cells were labeled for 24 hours with 10 μM EdU or DMSO as a vehicle control. Cells were fixed and prepared according to the manufacturer's instructions, minus the Amplex Ultra red labeling. Hoechst 33342 was added at 5 μg/mL (Invitrogen H1399), and labeled cells were imaged on a Zeiss inverted fluorescence microscope. Four tiled images at 10× magnification were collected per each well of 3 biological replicates per condition and stitched together as a single image per well for analysis. EdU-labeled and total nuclei were counted in Fiji.

### Western Blotting

Western blotting was performed using a modified Novus Biological protocol (NB100-2220) with 10%, 12%, 14% or 4-20% gradient SDS-PAGE gels. Cells were collected and lysed with RIPA buffer plus PMSF protease inhibitor. Cell lysate was centrifuged at high speed for 10 minutes at 4° C., and the supernatant was transferred to a fresh tube. The total protein content was quantified with a Bradford assay, and a total of 20 ug of protein was mixed with Laemmli buffer (4% SDS, 5% 2-mercaptoethanol (BME), 20% glycerol, 0.004% bromophenol blue, 0.125 M Tris HCl, pH 6.8) and denatured for 10 minutes at 95° C. This was followed by wet transfer to PVDF immobilon membrane (Millipore IPFL00010) and blocked with Odyssey TBS blocking buffer (LiCor 927-50000). The following primary antibodies were used in the listed concentrations: LC3 (Novus Biologicals NB100-2220, 1:500) and GAPDH (Sigma Aldrich G9545, 1:10,000). The LiCor IR secondaries were used 1:10,000 or 1:20,000 (LiCor 926-32211, 926-68071 and 926-32210). Bands were quantified and normalized by a ratio of LC3-II:GAPDH, rather than LC3-II:LC3-I as LC3-II is more sensitive in blotting and accurate observed alone [25].

### MitoTracker Staining

MitoTracker red (Thermo Fisher M22425) and green (Thermo Fisher M7514) were purchased from Thermo Fisher. MitoTracker red was diluted to 500 nM and MitoTracker green to 200 nM in Vasculife VEGF, along with Hoechst 33342 at 5 μg/mL (Invitrogen H1399). Live cells were incubated with the diluted dyes for 30 minutes at 37 degrees C. in the dark in a humidified 5% CO2 incubator. Cells were then washed with 1×PBS and fresh media was replaced. Cells were then imaged at 20X on a Zeiss inverted fluorescence microscope, 5 high power fields for each of 3 biological replicates, as well as an unstained control.

### Transmission Electron Microscopy

Therminox coverslips (Ted Pella 26028) were sterilized with 70% ethanol and UV irradiation for 1 hour prior to coating in the usual fashion (Matrigel for Y6 iPSCs; vitronectin for ACS1028 iPSCs; and fibronectin for iPSC-ECs). At the desired time intervals, the coverslips were removed and placed in warmed (0.1M sodium cacodylate, pH 7.4, 2% paraformaldehyde, 2.5% gluteraldehyde) and fixed for 60 minutes at 37 degrees. Cells were then post-fixed in 2% osmium tetraoxide in imidazole buffer 0.1M (pH 7.5) for 1 hour, rinsed in distilled water, stained with 3% UA for 1 hour, and again rinsed in distilled water. Samples were then dehydrated in ascending grades of acetone, acetone and resin, embedded in a mixture of an Embed 812 kit (EMS 14120), and cured for 48 hours in a 60° C. oven.

Ultrathin sections (70 nm) of the organoid were produced using a Leica UltraCut UC6 ultramicrotome, collected on 200 mesh copper grids and post stained with 3% UA and Reynolds lead citrate. Micrographs were collected using on a FEI Technai Spirit G2 TEM.

### Nitric Oxide Synthase Activity Assay

Nitric oxide synthase activity was measured using the DAF-FM kit from Thermo fisher (part number D23842) and read on a Syngene microplate reader in triplicate with an unlabeled control. iPSC-ECs were plated on vitronectin coated 96-well plates at 25,000 cells/cm 2. After 24 hours, cells were labeled with 10 uM DAF-FM and Hoechst 33342 at 5 μg/mL (Invitrogen H1399) in 1×PBS with calcium and magnesium for 30 minutes in a dark 5% CO2 humidified incubator, washed with 1×PBS, and allowed to de-esterify intracellular diacetates for an additional 30 minutes in a dark 5% CO2 humidified incubator. Signal at 488 nm and 360 nm were quantified in a Syngene plate reader.

Mitophagy and autophagy induction Bafilomycin A1 was purchased from Sigma Aldrich (#B 1793) and reconstituted in DMSO. Antimycin A1 was purchased from Sigma (#A8674) and reconstituted in ethanol. Oligomycin was purchased from Sigma (#75351) and reconstituted in DMSO. FCCP was purchased from Abcam (#ab120081) and reconstituted in DMSO. ULK1 kinase inhibitor SBI-0206965, known to inhibit autophagy, was purchased from Selleck Chemicals (#S7885) and reconstituted in DMSO [8]. Rapamycin was purchased from MP Biomedicals (#159346) and reconstituted in DMSO. Resveratrol was purchased from Sigma (R5010) and reconstituted in DMSO. ML246 and Rg2 were kindly provided by the lab of Dr. Congcong He and reconstituted in DMSO [9, 10].

### Data Analysis and Statistics

There were three biological replicates for each group in each experiment, unless otherwise specified. For MitoTracker quantification, total fluorescence signal for the area was measured in Fiji and divided by the number of nuclei present. This was done for 5 fields of view (technical replicates) and averaged to generate a data point for a given biological replicate. For the DAF-FM reduction measurements for NO production, a given experimental condition was plated into 3 wells of a 96 well plate. For TEM analysis, each biological sample was imaged in a minimum of 5 locations. Three representative images from each condition were utilized for quantifying autolysosomes per high power field (HPF) in each of two separate experiments. For light microscopy images, cells were manually counted from three distinct areas of a given experimental condition on two separate occasions. All data wereanalyzed with one-way ANOVA with a post-hoc Tukey test in IBM SPSS Statistics for Macintosh, version 27 (IBM Corp., Armonk, N.Y., USA). A p value of <0.05 was considered significant, where * represent comparisons between the specified groups.

### Results

iPSCs Successfully Differentiate into Endothelial Cells but Rapidly Lose Key Phenotypic Markers and Proliferative Capacity after a Few Passages

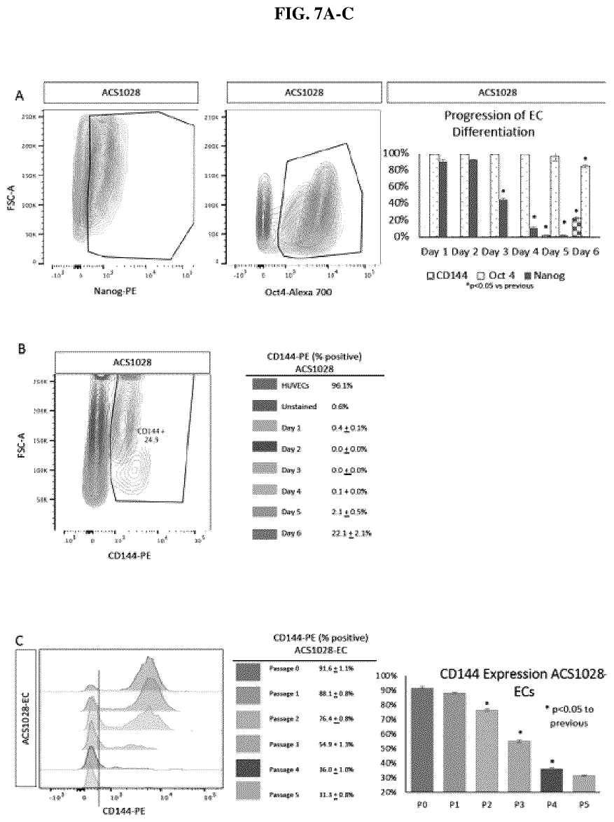

To validate the pluripotency of the iPSC lines, positive immunostaining of lines ACS1028 and Y6 for pluripotency markers was demonstrated by flow cytometry (FIG. 7A). Both iPSC lines were differentiated to endothelial cells using the protocol by Patsch, et al, occurring over 6 days, passing through lateral mesoderm in the first 3 days [7]. To verify the success of the differentiations, expression of endothelial cell markers CD144 and CD31 was measured, as well as expression of pluripotency markers on each of the 6 days of differentiation by immunostaining and by flow cytometry, with differentiation efficiencies ranging from 20% to 60% CD144+ cells per experiment (FIG. 7A-B). Pluripotency marker NANOG disappeared by Day 5 and pluripotency marker OCT4 began to decrease on Day 4 indicating directed differentiation to endothelial cells in a similar progression to Patsch et al [7]. This order has been suggested as NANOG likely plays a role in modulating Oct4 as a downstream gene [26]. On day 6 the endothelial cells were purified by magnetic associated cell sorting (MACS) for CD144. The resulting endothelial cells demonstrated normal spindle morphology and produced nitric oxide. These results demonstrate the successful loss of pluripotency and acquisition of mature endothelial cell markers and function over the course of the directed differentiation protocol.

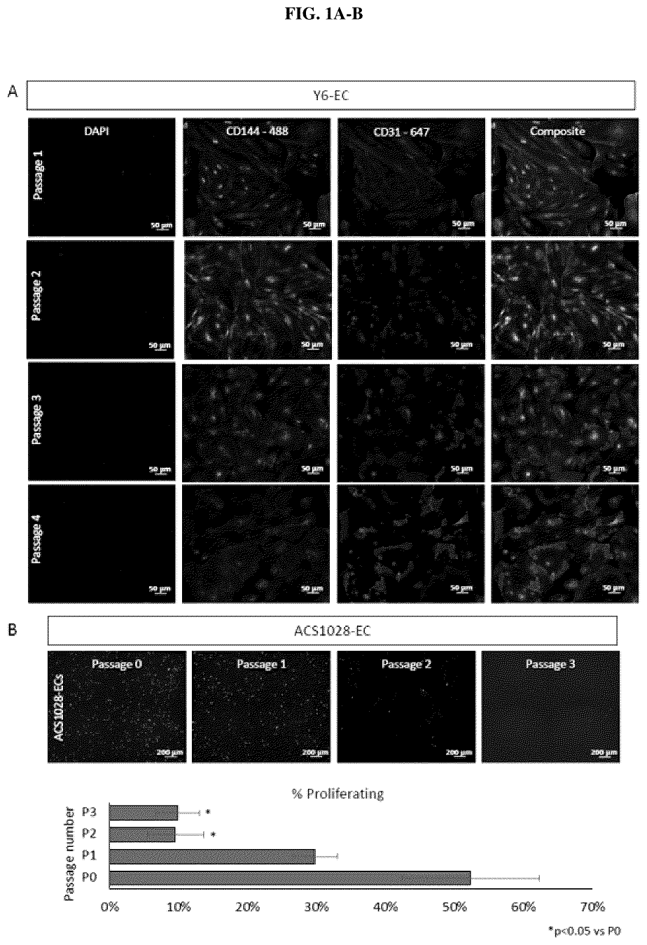

To evaluate endothelial cell function over serial passages in culture, typical morphology was examined, as well as CD144 and CD31 expression and proliferation. Immunofluorescence confirmed the loss of morphology as well as CD144 and CD31 expression over serial passages in culture for both Y6-derived ECs and ACS1028-derived ECs (FIG. 1A). At passage 2, CD144 expression was already decreased, 92% to 76%, finally nadiring at 30% in passages 4 and 5 (FIG. 7C). To evaluate cellular proliferation, iPSC-ECs were labeled with Edu, which is incorporated into replicating DNA and can be fluorescently labeled through click chemistry. iPSC-derived endothelial cell proliferation decreased significantly over serial passages, from 52% staining positive for 5-ethynyl-2′-deoxyuridine (EdU) to 30% from passage 0 to passage 1, nadiring at 10% in subsequent passages (FIG. 1B). The iPSC-derived endothelial cells were unable to be sub-cultured beyond 4 weeks, more commonly only for 3-4 passages over 2 weeks, post cell sorting due to replicative senescence (loss of proliferation by passage 3 shown in FIG. 1B). These results demonstrate loss of mature endothelial cell markers and proliferative capacity over just a few cultures in passage.

Autophagy Plays an Active Role During Directed Differentiation of iPSCs to ECs by Recycling Mitochondria Through Mitophagy

Senescence in iPSC-ECs can be attenuated by overexpression of sirtuinl on a lentivirus [5]. To facilitate future translational applications for iPSC-ECs, experiments were conducted to identify small molecules that enhance longevity of iPSC-ECs. Autophagy, a known downstream target of sirtuinl signaling and mediator of cellular senescence, was investigated. Autophagy is the process of cellular self-digestion to remove defective intracellular components and recycle them. This process occurs by engulfing the content to be degraded in the autophagosome, which then fuses with a lysosome to form an autolysosome, leading to the degradation of the enclosed contents. A key step in the formation of the autophagosome is the conversion of the cytosolic form of microtubule-associated proteins 1A/1B light chain 3B (LC3-I) to the lipidated autophagosome-associated form LC3-II by post-translational modification. The drug bafilomycin A1 is utilized to optimize detection of autophagy induction as it prevents fusion of the autophagosome to the lysosome, thereby blocking the degradation of newly formed LC3-II and other autophagosome contents by lysosomes.

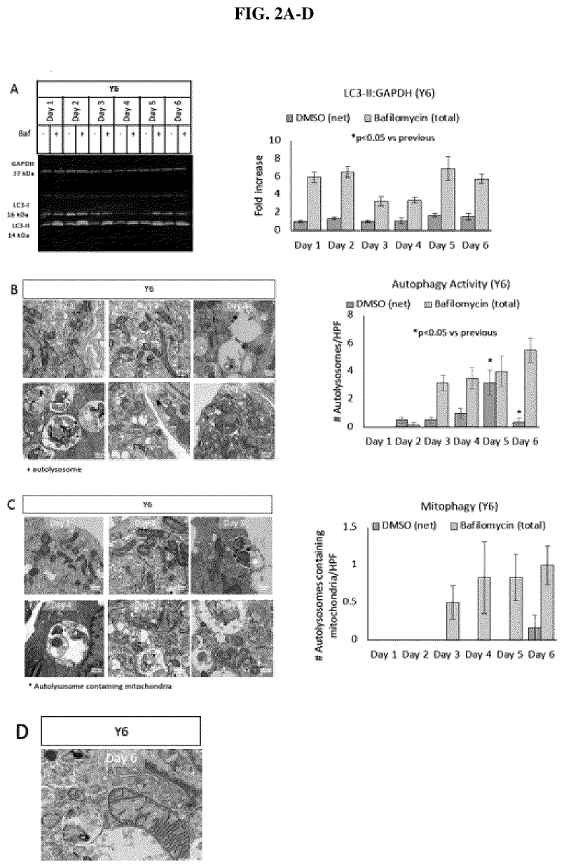

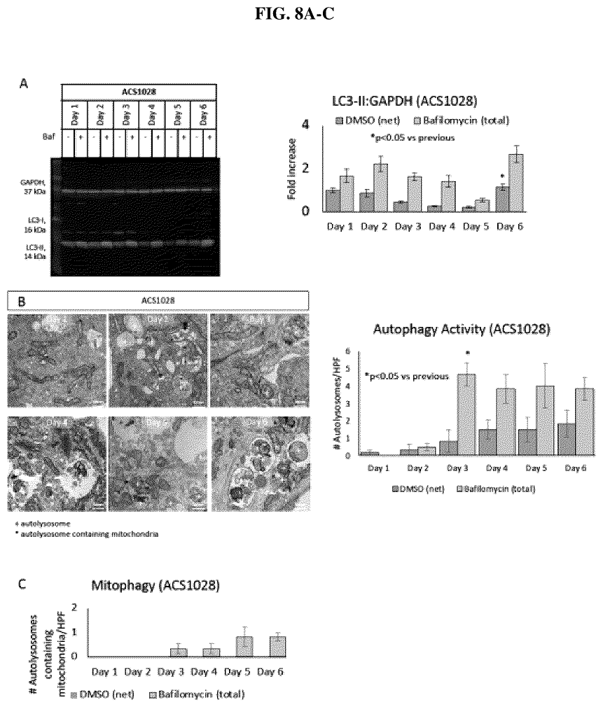

To understand the role autophagy plays in directed differentiation, LC3-II levels were quantified in iPSCs each of the 6 days during differentiation to ECs by Western blotting whole cell lysates in the presence or absence of 100 nM bafilomycin A1 for 3 hours. Optimal bafilomycin treatment was determined empirically for each iPSC line utilized. LC3-II expression normalized to GAPDH was used for quantitation, as it is a more reliable marker of autophagosome formation. LC3-I expression was noted to be lower than LC3-II and was sometimes not visible in the linear range for LC3-II. This may not be indicative of a lost signal, but rather a lower signal in general, as LC3-I signal is not optimally sensitive in blotting [25]. Therefore, the LC3-II signal, normalized to GAPDH, was measured each day of differentiation. The difference between LC3-II levels without bafilomycin A1 relative to total LC3-II levels reflects autophagosome turnover (flux), whereas high total LC3-II levels represent high autophagosome formation (induction). The two iPSC lines varied slightly in the pattern of autophagy flux and autophagy induction over the course of differentiation. Overall, both lines experienced small, though not statistically significant fluctuations in LC3-II formation (FIG. 2A, 8A). However, by TEM imaging in bafilomycin A1 treated Y6 cells, autolysosomes significantly increased in number from day 2 to day 3 by 19-fold (p<0.05) and remained abundant through day 6 (FIG. 2B). These data indicate dynamic autophagy activity during differentiation of Y6 iPSCs to ECs. In the ACS1028 line, autolysosomes similarly increased>9-fold in number on day 2 (p<0.05) and remained numerous through day 6 (FIG. 8B). The fluctuations in LC3-II levels and autolysosome number in multiple iPSC lines indicate that autophagy is highly dynamic and plays a crucial role during directed differentiation of iPSCs to ECs.

Mitophagy, the process by which autophagy engulfs and turns over mitochondria [11], contributes to cell fate reassignment in the process of generating iPSCs from terminally differentiated cells [12]. To assess the role mitophagy plays here in the opposite process of differentiating iPSCs to ECs, the number of autolysosomes containing mitochondria was quantified, which is indicative of active mitophagy, on each of the different days of differentiation. By TEM, multiple mitochondria were visualized within autolysosomes on days 3 through 6, but none on days 1 and 2 in both iPSC lines (FIG. 2B-C and FIG. 8B-C). Furthermore, mitochondrial morphology started round, immature and small on day 1, but by day 6 the mitochondria were long and filamentous with obvious cristae (representative image in FIG. 2D). Together these data demonstrate that mitophagy contributes to major changes in mitochondrial remodeling during differentiation, particularly during the late mesoderm phase and the committed phase from days 3-6.

iPSC-ECs demonstrate decreased mitophagy over serial passages in culture

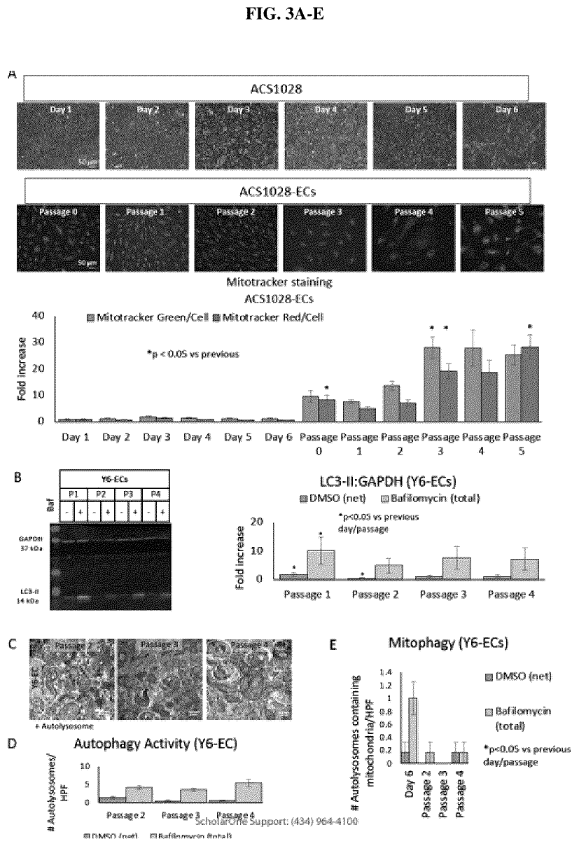

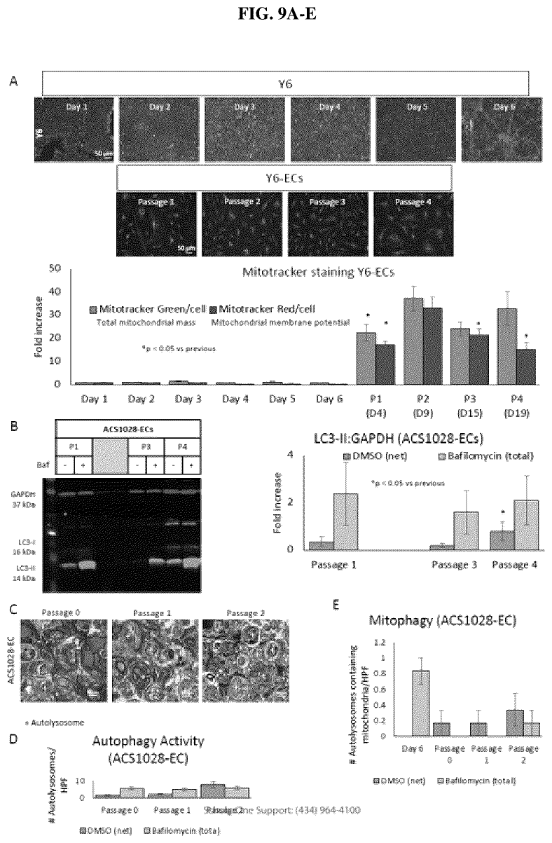

To further evaluate the effects of mitophagy on the mitochondria, changes in mitochondrial morphology and quantity were monitored by performing mitochondrial staining on each day of differentiation. MitoTracker signal was measured by live cell fluorescence microscopy to quantity mitochondrial mass on each day of differentiation. This enabled us to correlate serial changes in mitochondrial remodeling with autophagy activity. Two stains were utilized, red and green, to independently verify the mitochondrial quantity. Fluorescence signals were quantified and averaged on a per cell basis. Both iPSC lines experienced small not statistically significant fluctuations in MitoTracker staining during the course of differentiation (FIG. 3A and FIG. 9A, top and bottom panels each).

To evaluate the role of mitophagy in purified iPSC-ECs over serial passages, mature iPSC-derived ECs were also stained with MitoTracker red and green. For both ACS1028-derived ECs and Y6-derived ECs, there was a significant accumulation of mitochondria with a greater than 20-fold increase over serial passages (p<0.05) (FIG. 3A and FIG. 9A, middle and bottom panels each). Specifically, purified iPSC-ECs (ACS1028) demonstrated a 13- and 28-fold increase in total mitochondrial mass at passages 2 and 3, respectively, compared with day 6 of the directed differentiation. This increase remained stable through passage 5, at which time the cells demonstrated replicative senescence. In the Y6-derived ECs, MitoTracker staining increased greater than 20-fold at passage 1 and remained increased through passages 3 and 4. At this time the cells demonstrated replicative senescence.

Mitophagy is known to play a critical role in stem cell differentiation [13]. To further evaluate whether this accumulation of mitochondria may be a failure of mitophagy, autophagy activity was measured over serial passages. In Y6-derived endothelial cells, the total LC3-II level increased at passage 1 but then decreased and remained steady at levels comparable to the level on day 6 (FIG. 3B). The total LC3-II level plateaued and remained stable compared to the level detected on day 6 in ACS1028-derived endothelial cells (FIG. 9B). Similarly, the total number of autolysosomes remained steady over serial passages in both lines (FIG. 3C-D, FIG. 9C-D). Autolysosomes containing mitochondria were absent at later passages despite a comparable number of total autolysosomes (FIG. 3E, FIG. 9E). These findings indicate that autophagy, more specifically, mitophagy, is not recycling mitochondria in later passages.

Hypoxia Reduces but does not Eliminate Mitochondrial Accumulation

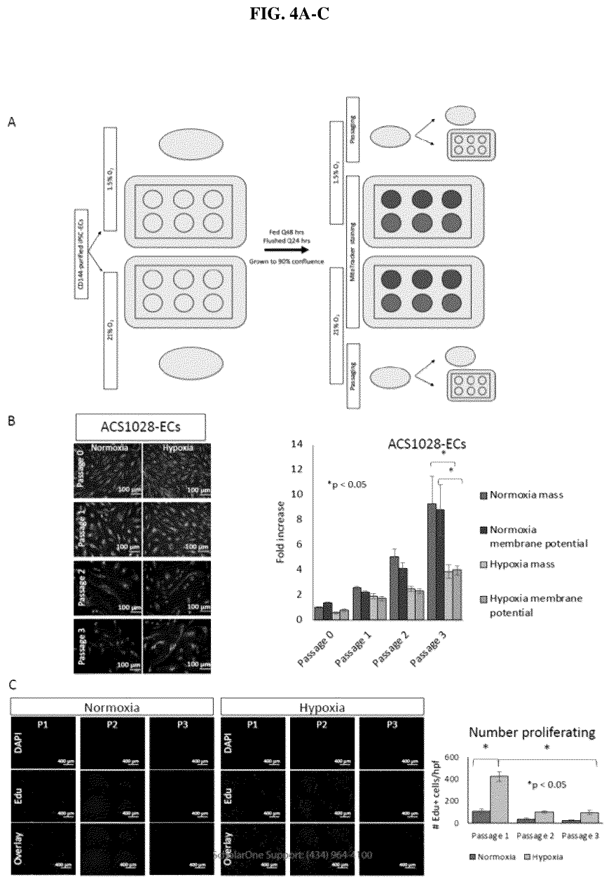

Given that oxidative damage leads to mitochondrial dysfunction [11], Experiments were conducted to determine whether supraphysiologic oxygen concentration of standard culture conditions was contributing to mitochondrial accumulation and to the premature senescence phenomena. To evaluate the effect of hypoxia on mitochondrial accumulation, iPSC-ECs were cultured under normoxic (21%) and hypoxic (1.5%) conditions. A schematic diagram of the experimental design is illustrated in FIG. 4A. Each passage was stained with MitoTracker, green and red, and imaged (FIG. 4B, 10B top row). Signal intensity per cell was calculated and normalized to MitoTracker green staining from normoxic cells at passage 0 for each cell line. Cells cultured in hypoxic conditions demonstrated decreased mitochondria per cell by MitoTracker green and red staining when compared with cells grown under normoxic conditions at the same passage, which reached statistical significance in later passages (FIG. 4B, 10C). This was true for iPSC-derived ECs from both ACS1028 and Y6 lines. Both lines, however, continued to demonstrate an increase in mitochondria per cell over serial passages, although less so under hypoxic conditions. These results demonstrate that hypoxia decreases but does not eliminate the accumulation of mitochondria over serial passages of iPSC-ECs.

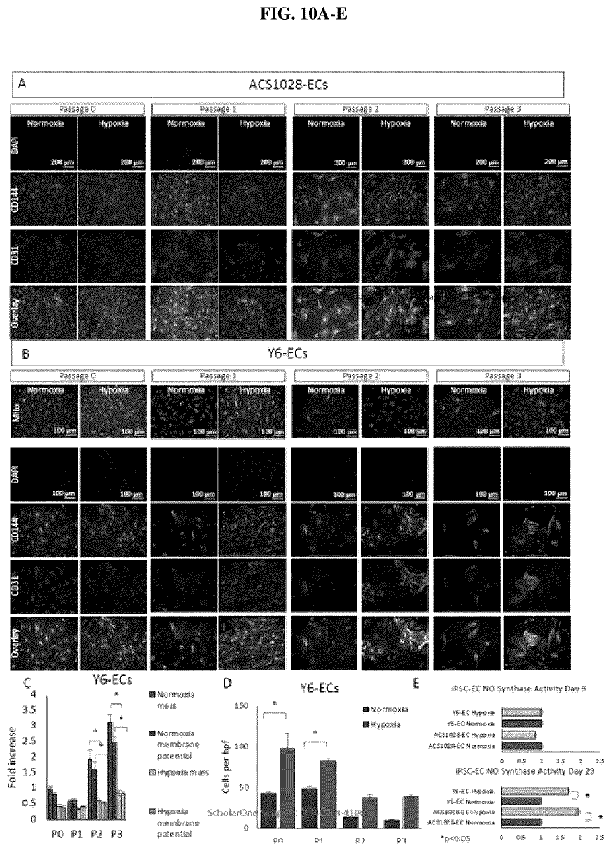

To assess the effect of hypoxia on proliferation, cells were labeled at each passage with EdU. This demonstrated an increase in the number of proliferating cells at passage 1 under hypoxic conditions as compared with normoxia in ACS1028-ECs, but this was not sustained over serial passages (FIG. 4C). Similarly, for Y6-ECs, nuclei were counted per high power field over 5 areas for each of 3 biological replicates, which demonstrated an increased number of cells under hypoxic conditions compared with normoxic conditions in initial passages that did not sustain over serial passages (FIG. 10D). Cell numbers did still diminish in later passages under both conditions for both cell lines. These results demonstrate that hypoxia improves proliferation and survival, but iPSC-ECs still reach replicative senescence even under hypoxic conditions.

iPSC-EC function was further evaluated under hypoxic conditions by examining morphology and staining for key markers CD144 and CD31, as well as assessing NO production. While the surviving cells did express CD144 and CD31 by immunostaining, the cells still demonstrated the same loss of typical morphology with non-uniform membrane staining at later passages under both normoxic and hypoxic conditions, which indicates that the cells still lost mature cell properties even under hypoxic conditions (FIG. 10A, 10B lower rows). NO synthase activity did not differ at early passages between normoxic and hypoxic conditions but was preserved at later passages with hypoxic compared to normoxic conditions (FIG. 10E). Taken together, these results demonstrate that hypoxic conditions improve but do not eliminate mitochondrial accumulation and loss of cellular proliferation and mature phenotype.

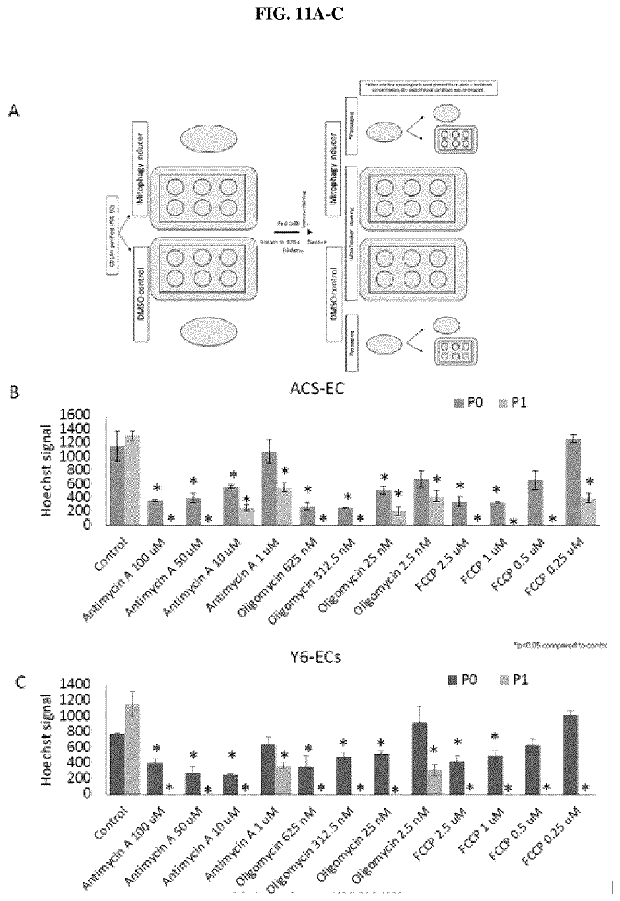

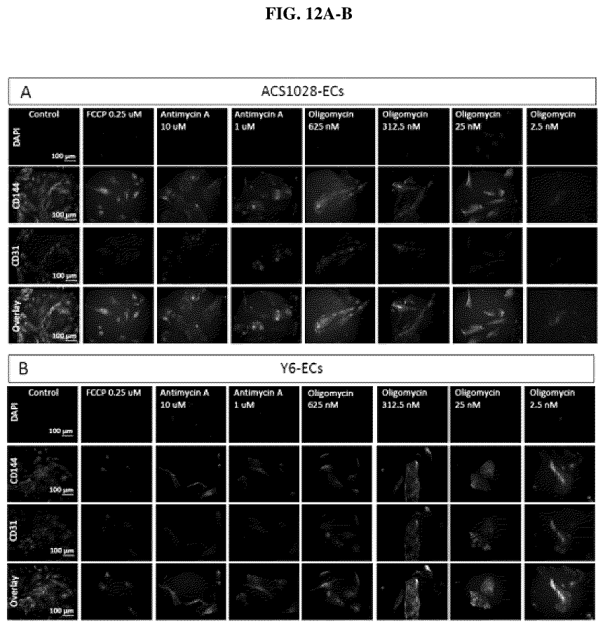

The effect of increasing mitophagy in iPSC-ECs was evaluated. ACS1028-ECs were cultured in the presence or absence of several different concentrations of known inducers of mitophagy, Antimycin A and oligomycin. The experimental design is presented in FIG. 11A. Mitophagy inducers Antimycin A and oligomycin resulted in high levels of cell loss, regardless of concentration (FIG. 11B-C). This was also true of mitochondrial uncoupler FCCP, indicating that iPSC-ECs do not tolerate the loss of mitochondrial membrane potential. Surviving cells rapidly lost their CD144 and CD31 uniform membrane staining by passage 1 (FIG. 12A-B), which indicates that direct pharmacologic stimulation of mitophagy through membrane depolarization accelerated rather than alleviated the senescence problem.

mTOR-Independent ULK1-Kinase Mediated Autophagy Improves iPSC-EC Proliferation

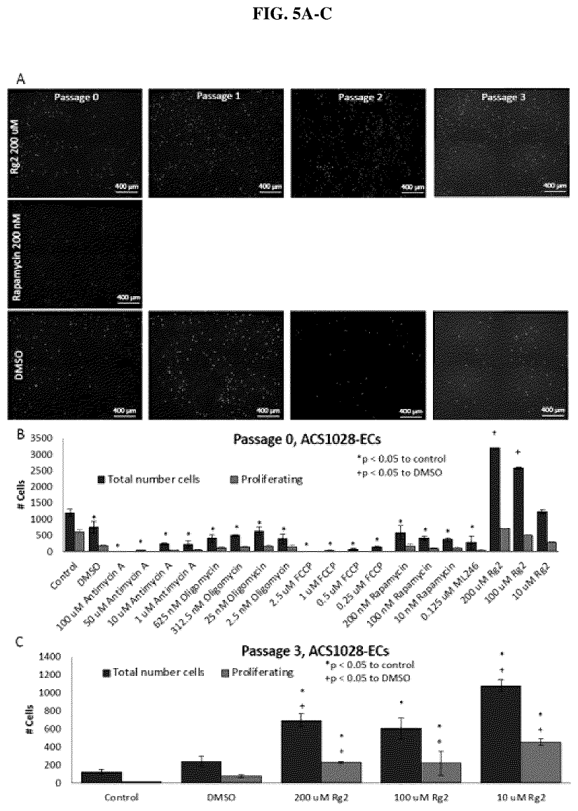

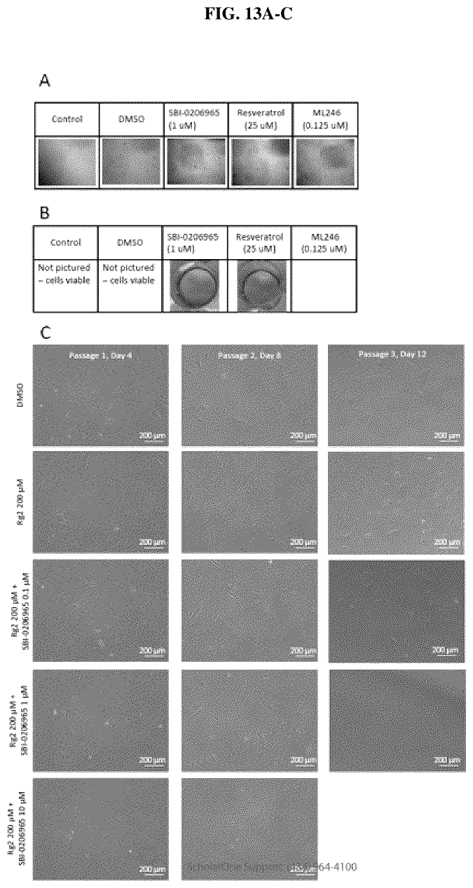

Experiments were conducted to determine whether inducing autophagy in iPSC-ECs overcomes the premature senescence phenomenon. Both autophagy inducer resveratrol and autophagy inhibitor SBI-0206965 impeded cell survival, with no cells surviving to passage 1. Autophagy inducer ML246 [9] yielded no viable cells after 24 hours at the higher concentrations of 0.5 μM and 0.25 μM. A reduced concentration of 0.125 μM still yielded too few cells to passage (FIG. 13A-B). Rapamycin, at all tested concentrations, also yielded an insufficient number of cells for passaging (FIG. 5A-B). Rg2, an mTOR-independent inducer of autophagy [10], improved cell survival with an increased rate of proliferation (FIG. 5A-C). Due to the loss of proliferating cells in the control culture, the experiment was terminated at passage 3. The effect of Rg2 persisted through passage 3, although the number of total and of proliferating cells were fewer after 4 days in culture at passage 3 than at passage 0 (FIG. 5C). As Rg2 is known to be mTOR-independent, and rapamycin did not produce any beneficial results to proliferation, these results indicate that Rg2 produces a beneficial effect on iPSC-EC longevity through an mTOR-independent pathway.

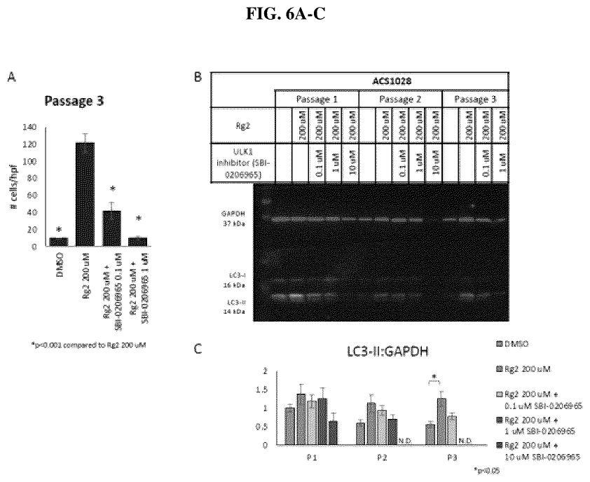

To assess whether the effect of Rg2 on proliferation is mediated through autophagy, ACS1028-ECs was administered with 200 μM Rg2 and SBI-0206965 to block ULK1 kinase at varying concentrations. Cell survival was assessed by counting the number of cells per high power field by light microscopy, which diminished over serial passages in the DMSO-treated vehicle-control condition. Decreased cell survival to passage 3 demonstrated a dose-dependent response to SBI-0206965 in the presence of 200 μM Rg2, with fewer cells surviving when treated with higher concentrations of SBI-0206965 (FIG. 6A, FIG. 13C). At the highest concentration of SBI-020965 used, 10 μM, there were almost no surviving cells at passages 2 and 3. At passage 3, the Rg2 treated cells demonstrated an increase in LC3-II level by Western blotting, which was reduced by the addition of ULK1 kinase inhibitor SBI-0206965 at any concentration (FIG. 6B-C), indicating that the longevity-enhancing increased LC3-II level was mediated by ULK1 kinase autophagy.

### Example 2

Experiments conducted during development of embodiments herein demonstrate that administration of an AMPK activator, specifically the ginsenoside Rg2 to human induced pluripotent stem cell-derived endothelial cells results in: (1) increased expression of longevity protein SIRT1 in a dose-dependent manner; (2) increased phosphorylation (e.g., activity) of endothelial nitric oxide synthase; (3) increased Parkin expression (a marker of mitophagy), particularly under bafilomycin treated conditions, to inhibit autophagosome-lysosome fusion; (4) increased phosphorylation of Drp1 (mitochondrial fission) and expression of Opal (mitochondrial fusion); and (5) increased phosphorylation of PKM2 and expression of PDH, particularly under bafilomycin treated conditions, to inhibit autophagosome-lysosome fusion. Rg2 administration did not induce apoptosis (no increase in cleaved caspase 3 at tested doses).

### Western Blotting Procedure

Utilizing 8%, 10%, and 14% polyacrylamide gels, samples were run at 150V for 1 to 2 hours in SDS-Tris-Glycine buffer. Gels were then transferred to nitrocellulose membrane for two and half hours at 0.35 Amps in Tris-Glycine transfer buffer. After transfer nitrocellulose membranes were blocked in 10% milk/TBST solution for 1 hr. Milk solution was removed, and membranes rinsed in TBST. Blocked membranes were cut and placed in primary antibody solution (5% BSA/TBST) overnight. After incubation with primary antibody incubation membranes were washed with TBST for 5 mins 3X. Secondary antibody incubation (Rabbit 1:1000) in 5% BSA solution was applied for 2 hrs. After secondary antibody incubation membranes were washed with TBST for 15 mins 3X. ECL was applied for chemiluminescence. Imaging was done using Bio-Rad imager.

p-enos Y1177CST 9570, 1:1000
sirt 1 Thermo Fisher PAS-17074, 1:1000
parkin Proteintech 14060-1-AP, 1:1000
caspase3 CST 9662, 1:1000
p-drp1 CST 3455, 1:1000
opal CST 67589, 1:1000

### REFERENCES

The following references, some of which are cited herein by number, are herein incorporated by reference in their entireties.
1. Banito A, Gil J. Induced pluripotent stem cells and senescence: learning the biology to improve the technology. EMBO Rep 2010; 11(5):353-9.
2. Menendez JA, Vellon L, Oliveras-Ferraros C, et al. mTOR-regulated senescence and autophagy during reprogramming of somatic cells to pluripotency: a roadmap from energy metabolism to stem cell renewal and aging. Cell Cycle 2011; 10(21):3658-77.
3. Li Z, Hu S, Ghosh Z, et al. Functional characterization and expression profiling of human induced pluripotent stem cell- and embryonic stem cell-derived endothelial cells. Stem Cells Dev 2011; 20(10): 1701-10.
4. Wimmer RA, Leopoldi A, Aichinger M, et al. Human blood vessel organoids as a model of diabetic vasculopathy. Nature 2019; 565(7740):505-10.
5. Jiang B, Jen M, Perrin L, et al. SIRT1 Overexpression Maintains Cell Phenotype and Function of Endothelial Cells Derived from Induced Pluripotent Stem Cells. Stem Cells Dev 2015; 24(23):2740-5.
6. Gokoh M, Nishio M, Nakamura N, et al. Early senescence is not an inevitable fate of human-induced pluripotent stem-derived cells. Cell Reprogram 2011; 13(4):361-70.
7. Patsch C, Challet-Meylan L, Thoma E C, et al. Generation of vascular endothelial and smooth muscle cells from human pluripotent stem cells. Nat Cell Biol 2015; 17(8):994-1003.
8. Egan DF, Chun MG, Vamos M, et al. Small Molecule Inhibition of the Autophagy Kinase ULK1 and Identification of ULK1 Substrates. Mol Cell 2015; 59(2):285-97.
9. Rocchi A, Yamamoto S, Ting T, et al. A Becnl mutation mediates hyperactive autophagic sequestration of amyloid oligomers and improved cognition in Alzheimer's disease. PLoS Genet 2017;13(8):e1006962.
10. Fan Y, Wang N, Rocchi A, et al. Identification of natural products with neuronal and metabolic benefits through autophagy induction. Autophagy 2017; 13(1):41-56.
11. Lee J, Giordano S, Zhang J. Autophagy, mitochondria and oxidative stress: cross-talk and redox signalling. The Biochemical journal 2012; 441(2):523-40.
12. Ma T, Li J, Xu Y, et al. Atg5-independent autophagy regulates mitochondrial clearance and is essential for iPSC reprogramming. Nat Cell Biol 2015; 17(11):1379-87.
13. Sotthibundhu A, Promjuntuek W, Liu M, et al. Roles of autophagy in controlling stem cell identity: a perspective of self-renewal and differentiation. Cell Tissue Res 2018.
14. Zhou J, Su P, Wang L, et al. mTOR supports long-term self-renewal and suppresses mesoderm and endoderm activities of human embryonic stem cells. Proceedings of the National Academy of Sciences of the United States of America 2009; 106(19):7840-5.
15. Sotthibundhu A, McDonagh K, von Kriegsheim A, et al. Rapamycin regulates autophagy and cell adhesion in induced pluripotent stem cells. Stem Cell Res Ther 2016;7(1):166.
16. Zhou J, Sears RL. Bioinformatics Approaches to Stem Cell Research. Current Pharmacology Reports 2018; 4(4):314-25.
17. Bukys MA, Mihas A, Finney K, et al. High-Dimensional Design-Of-Experiments Extracts Small-Molecule-Only Induction Conditions for Dorsal Pancreatic Endoderm from Pluripotency. iScience 2020;23(8):101346.
18. Kitano H. Computational systems biology. Nature 2002;420206-10.
19. Carinhas N, Oliveira R, Alves P M, et al. Systems biotechnology of animal cells: the road to prediction. Trends Biotechnol 2012; 30(7):377-85.
20. Dai DF, Danoviz ME, Wiczer B, et al. Mitochondrial Maturation in Human Pluripotent Stem Cell Derived Cardiomyocytes. Stem Cells Int 2017;20175153625.
21. Korski KI, Kubli DA, Wang B J, et al. Hypoxia Prevents Mitochondrial Dysfunction and Senescence in Human c-Kit(+) Cardiac Progenitor Cells. Stem Cells 2019; 37(4):555-67.
22. Guan JL, Simon AK, Prescott M, et al. Autophagy in stem cells. Autophagy 2013; 9(6):830-49.
23. Jin SM, Youle RJ. PINK1- and Parkin-mediated mitophagy at a glance. J Cell Sci 2012; 125(Pt 4):795-9.
24. Xing J, Liu H, Yang H, et al. Upregulation of Unc-51-like kinase 1 by nitric oxide stabilizes SIRT1, independent of autophagy. PLoS One 2014;9(12):e116165.
25. Mizushima, N. & Yoshimori, T. How to interpret LC3 immunoblotting. Autophagy 3, 542-545 (2007).
26. Loh, Y.-H. et al. The Oct4 and Nanog transcription network regulates pluripotency in mouse embryonic stem cells. Nat Genet 38, 431-440 (2006).
27. Lamichane, S. et al. MHY2233 Attenuates Replicative Cellular Senescence in Human Endothelial Progenitor Cells via SIRT1 Signaling. Oxidative Medicine and Cellular Longevity 2019, e6492029 (2019).
28. Guerrero, F. et al. Passage Number-Induced Replicative Senescence Modulates the Endothelial Cell Response to Protein-Bound Uremic Toxins. Toxins (Basel) 13, 738 (2021).

1. A method of increasing the longevity of a cell population comprising stimulating mTOR-independent ULK1-mediated autophagy in the cells.

2. The method of claim 1, wherein the cell population comprises cells derived from induced pluripotent stem cells (iPSCs).

3. The method of claim 2, wherein the cell population comprises induced pluripotent stem cell-derived endothelial cells (iPSC-ECs).

4. The method of claim 1, comprising administering an agent to the cell population to stimulate mTOR independent ULK1-mediated autophagy.

5. The method of claim 4, wherein the agent is a small molecule.

6. The method of claim 4, wherein the cell population is cultured in the presence of the agent.

7. The method of claim 4, wherein the agent is an adenosine monophosphate kinase (AMPK) activator.

8. The method of claim 7, wherein the AMPK activator is an indirect activator.

9. The method of claim 8, wherein the indirect activator is Rg2.

10. The method of claim 7, wherein the AMPK activator is a direct activator.

11. The method of claim 4, wherein the agent is an activator of unc-51-like autophagy activating kinase 1 (ULK1).

12. A cell population comprising cells that have been cultured in the presence of an agent capable of increasing the longevity of a cell population comprising stimulating mTOR-independent ULK1-mediated autophagy.

13. The cell population of claim 12, wherein the agent is an adenosine monophosphate kinase (AMPK) activator.

14. The cell population of claim 12, wherein the agent is an activator of unc-51-like autophagy activating kinase 1 (ULK1).

15. The cell population of claim 12, wherein the cells are iPSC-derived cells.

16. The cell population of claim 15, wherein the cells are iPSC-ECs.

17. A medical device comprising cells of the cell population of claim 12.

18. The medical device of claim 17, wherein the device is a vascular graft.

19. A bioengineered organ comprising cells of the cell population of claim 12.

20. A method of treating a condition in a subject comprising administering cells of the cell population of claim 12 to a subject.

21. The method of claim 20, wherein the condition is ischemic tissue and iPSC-ECs are administered to promote angiogenesis.

22. A composition comprising (a) cell growth media components and (b) and agent capable of stimulating mTOR-independent ULK1-mediated autophagy in the cells.
